(19) United States
(12) Patent Application Publication
(10) Pub. No.: US 2003/0175233 A1
(43) Pub. Date: Sep. 18, 2003

(54) Hair cosmetic agent

(76) Inventors: Peter Hossel, Schifferstadt (DE); Reinhold Dieing, Schifferstadt (DE)

Correspondence Address: KEIL & WEINKAUF, 1350 CONNECTICUT AVENUE, N.W., WASHINGTON, DC, 20036, US

(21) Appl. No.: 10/275,567

(22) PCT Filed: May 3, 2001
(86) PCT No.: PCT/EP01/04962

(30) Foreign Application Priority Data
May 12, 2000 (DE) 100 23 245.0

(51) Int. Cl. A61K 7/06; A61K 7/11

(52) U.S. Cl. 424/70.17

(57) ABSTRACT

Hair cosmetic agents which comprise a polymer and a relaxer are proposed. The agents according to the invention are suitable for effective hair smoothing whilst being gentle on the scalp, and for retaining the structure of the hair.

The invention is in the field of cosmetic agents and relates to hair-treatment agents.

### PRIOR ART

Plastic deformation of hair is made possible by the partial reduction of the disulfide bridges in the keratin of hair. Here, it is necessary to differentiate between the process of permanent waving (1. partial reduction of the cystine to cysteine (=hair softening), 2. mechanical deformation 3. oxidative closure of the previously opened disulfide bridges), and the smoothing of naturally curly or artificially curled hair. In the case of very naturally curly hair (e.g. Afro-American hair) stylability and manageability is in most cases impossible without prior hair smoothing because of the many disulfide bridges in the hair. For hair smoothing, the hair is usually moistened with a hair-smoothing agent and then mechanically smoothed over and over again (e.g. by repeated combing). Depending on the concentration and strength of the hair, these preparations are left on the hair for varying amounts of time and rinsed out completely using a neutralizing solution. As a result of hair smoothing, the hair generally loses between 40 and 50% of its tensile force, presumably as a result of the structural damage caused by the hair-smoothing process. Three different types of preparations are known: hair-smoothing agents based on alkali metal hydroxides, hair-smoothing agents based on guanidine hydroxide, and hair-smoothing agents which have a reducing effect, such as, for example, thioglycolic acid.

Hair-smoothing preparations based on thioglycolic acid or sodium hydroxide are described, for example, in W. Umbach, 1988, Kosmetik (Cosmetics), Thieme Verlag, p. 263. Preparations based on alkali metal hydroxides, for example 1.8 to 2.5% strength by weight solutions of sodium hydroxide with a pH of 12-14 have a high irritation potential for the scalp and strongly attack the hair. Hair-smoothing agents based on guanidine hydroxide, as described, for example, in U.S. Pat. No. 4,314,572, are prepared in situ on the hair by mixing a calcium hydroxide solution with a guanidine carbonate solution. These agents damage the scalp and attack the hair. Although hair-smoothing agents which have a reducing action and are based on thioglycolates are milder to the scalp and hair, they are unsatisfactory in their effectiveness and, moreover, require long contact times. The chemical aggressiveness of the known hair-smoothing preparations requires application by hairdressers, accurate observation of contact times, careful formulation, and additional protection of the scalp and an aftertreatment to restore the damaged structure of the hair.

U.S. Pat. No. 5,060,680 and U.S. Pat. No. 5,148,822 describe quaternary ammonium compounds with a carbon chain length of from 11 to 18 carbon atoms which can be used after a hair-smoothing treatment. A disadvantage of these compounds is that they do not develop their action in combination with the hair-smoothing agent. Their use is therefore limited to aftertreatment shampoos.

U.S. Pat. No. 5,639,449 describes preparations which comprise 95 to 99.5% of a hair-aftertreatment agent (alkaline hair relaxer), and 5 to 0.5% of a condensation product from a C₁-C₃-dialkylamine, a difunctional epoxy component and a third reactant chosen from the group consisting of ammonia, primary amines, alkyldiamines having 2 to 6 carbon atoms and polyamines.

U.S. Pat. No. 5,565,216 describes a 2-component composition for the treatment of hair (relaxer). During use, the two components (cream base and activator) produce guanidine hydroxide in situ, which relaxes the hair. These compositions may comprise nonpolymeric quaternary nitrogen compounds as further constituents in the cream base.

EP 0 893 117 A2 describes the use of polymers obtainable by free-radically initiated copolymerization of

(a) 1 to 99.99% by weight of at least one cationic monomer or quaternizable monomer

(b) 0 to 98.99% by weight of at least one water-soluble monomer different from (a),

(c) 0 to 50% by weight of at least one further free-radically copolymerizable monomer different from (a) or (b) and

(d) 0.01 to 10% by weight of at least one bi- or polyfunctional free-radically copolymerizable monomer different from (a), (b) or (c) and

subsequent quaternization of the polymer if a nonquaternized monomer is used as monomer (a), as conditioning agents for hair.

Disadvantages of the known solutions are, as before, the irritation to the scalp caused during hair smoothing, and the unsatisfactory hair structure after hair smoothing, in particular the roughness, which is to be attributed to damage of the cuticle. Most known solutions require an aftertreatment with, for example, a care shampoo.

It is an object of the present invention to provide agents which permit effective deformation of hair (both for hair smoothing and also for deformation in the course of a permanent waving treatment) without irritating the scalp and damaging the hair in the process. The agents were to effect effective smoothing of tightly curled hair, irrespective of the condition of the hair. Furthermore, they were to have a temperature stability of up to 45° C. The agents were to be readily applied and usable without a change in consistency. The contact time of the preparations here should not be extended compared with the agents of the prior art. A gentle and rapid washing out of the hair-smoothing preparation using lukewarm water is desirable. It is also desirable that the hair feels light and soft, and has good stylability and, in particular, is additionally protected from thermal stress. This is of importance particularly in the case of preparations used in the course of a permanent wave treatment. Also desirable is good wet and dry combability, and reduced electrostatic charging. The preparations themselves were to be easy to handle from an applications viewpoint for the consumer, and were, in particular, to be usable as far as possible in one application step. This applies in particular to the known 2-component systems (active principle guanidine hydroxide); here the use of a further application step was to be avoided. The structure of the hair was to be permanently improved, and, in particular, the elasticity and tensile strength were to be retained. The agents were to be able to be prepared in stable formulations.

We have surprisingly found that this object is achieved by the agents according to the invention. They permit effective relaxation without irritation to the scalp occurring or without the hair becoming damaged. Surprisingly, both the relaxation itself and also the structure of the smoothed hair is improved. The hair treated in this way is readily combable and stylable and is protected from thermal stress. In particular, it is possible to dispense with an aftertreatment, e.g. a care shampoo.

### DESCRIPTION OF THE INVENTION

The invention provides hair cosmetic agents comprising

(i) polymer obtainable by free-radically initiated polymerization of

(a) 1 to 100% by weight, preferably 2 to 95% by weight, in particular 10 to 70% by weight, of at least one cationic monomer chosen from N-vinylimidazoles and diallylamines, optionally in partially or completely quaternized form,

(b) 0 to 99% by weight, preferably 5 to 98% by weight, in particular 30 to 90% by weight, of at least one water-soluble monomer different from (a) and

(c) 0 to 50% by weight, preferably 0 to 40% by weight, in particular 0 to 30% by weight, of at least one further free-radically copolymerizable monomer different from (a) or (b) and

subsequent partial or complete quaternization or protonation of the polymer if a nonquaternized or only partially quaternized monomer is used as monomer (a),

(ii) relaxer.

The invention further provides hair cosmetic agents comprising

(i) polymer obtainable by free-radically initiated copolymerization of

(a) 1 to 99.99% by weight, preferably 2 to 94.98% by weight, in particular 10 to 70% by weight, of at least one cationic monomer optionally in partially or completely quaternized form,

(b) 0 to 98.99% by weight, preferably 5 to 97.98% by weight, in particular 20 to 89.95% by weight, of at least one water-soluble monomer different from (a),

(c) 0 to 50% by weight, preferably 0 to 40% by weight, in particular 0 to 30% by weight, of at least one further free-radically copolymerizable monomer different from (a) or (b) and

(d) 0.01 to 10% by weight, preferably 0.02 to 8% by weight, in particular 0.05 to 5% by weight, of a di- or polyfunctional free-radically copolymerizable monomer different from (a), (b) or (c) and

subsequent partial or complete quaternization or protonation of the polymer if a nonquaternized or only partially quaternized monomer is used as monomer (a),

(ii) relaxer.

Polymer

Suitable as polymer component (i) are, for example, the polymers described in EP 0893 117 A2 and EP 246 580 B1, EP 544 158 B1 and EP 715 843 B1. They are available, for example, under the trade name Luviquat Care® (BASF). Also suitable are e.g. copolymers of vinylpyrrolidone/N-vinylimidazolium salts (Luviquat® FC, Luviquat® HM, Luviquat® MS), and copolymers of N-vinylcaprolactam/N-vinylpyrrolidone/N-vinylimidazolium salts (Luviquat® Hold). Such polymers are available under the INCI name Polyquaternium 16, Polyquaternium 40, Polyquaternium 44 and Polyquaternium 46.

Suitable monomers (a) are N-vinylimidazole derivatives of the formula (I) in which R¹ to R³ are hydrogen, C₁-C₄-alkyl or phenyl.

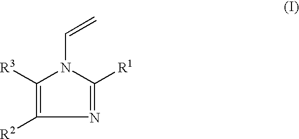

Examples of compounds of the formula (I) are given in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Further monomers of the formula (I) which can be used are the ethyl, propyl or butyl analogs of the methyl-substituted 1-vinylimidazoles listed in Table 1.

Also suitable are diallyamines [sic] of the formula (II) in which R⁴ is C₁-C₂₄-alkyl.

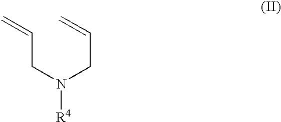

Examples of compounds of the formula (II) are diallylamines in which R⁴ is methyl, ethyl, iso- or n-propyl, iso-, n- or tert-butyl, pentyl, hexyl, heptyl, octyl, nonyl or decyl. Examples of longer-chain radicals R⁴ are undecyl, dodecyl, tridecyl, pentadecyl, octadecyl and icosayl [sic].

Also suitable are N,N-dialkylaminoalkyl acrylates and methacrylates and N,N-dialkylaminoalkylacrylamides and -methacrylamides of the formula (III),

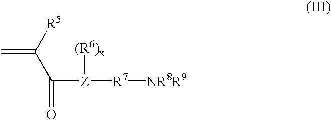

where R⁵, R⁶ independently are a hydrogen atom or a methyl radical, R⁷ is an alkaline radical having 1 to 24 carbon atoms, optionally substituted by alkyl radicals and R⁸, R⁹ are C₁-C₂₄-alkyl radicals. Z is a nitrogen atom together with x=1, or is an oxygen atom together with X=0.

Examples of compounds of the formula (III) are N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N,N-dimethylaminobutyl (meth)acrylate, N,N-diethylaminobutyl (meth)acrylate, N,N-dimethylaminohexyl (meth)acrylate, N,N-dimethylaminooctyl (meth)acrylate, N,N-dimethylaminododecyl (meth)acrylate, N-[3-(dimethylamino)propyl]methacrylamide, N-[3-(dimethylamino)propyl]acrylamide, N-[3-(dimethylamino)butyl]methacrylamide, N-[8-(dimethylamino)octyl]methacrylamide, N-[12-(dimethylamino)dodecyl]methacrylamide, N-[3-(diethylamino)propyl]methacrylamide, N-[3-(diethylamino)propyl]acrylamide.

Suitable for the quaternization of the compounds of the formula (I)-(III) are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic monomers of the formula (I)-(III) can also be carried out with alkylene oxides, such as ethylene oxide or propylene oxide, in the presence of acids.

The quaternization of the monomer (a) or of a polymer with one of said quaternizing agents can be carried out by generally known methods.

The quaternization of the polymers can be carried out completely or else only partially. The proportion of quaternized monomers (a) in the copolymer can vary over a wide range and is e.g. approximately 20 to 100 mol %.

Preferred quaternizing agents are: methyl chloride, dimethyl sulfate or diethyl sulfate.

Suitable for the protonation of, for example, mineral acids, such as HCl, H₂SO₄, H₃PO₄, and monocarboxylic acids, such as e.g. formic acid and acetic acid, dicarboxylic acids and polyfunctional carboxylic acids, such as e.g. oxalic acid and citric acid, and all other proton-donating compounds and substances which are able to protonate the corresponding vinylimidazole or diallylamine. In particular, water-soluble acids are suitable for the protonation.

The protonation of the polymer can either be carried out after the polymerization, or during the formulation of the cosmetic composition, during which a physiologically compatible pH is normally established.

Protonation is understood as meaning that at least some of the protonatable groups of the polymer, preferably 20 to 100 mol %, are protonated, such that an overall cationic charge of the polymer results.

In a preferred embodiment, as monomer (a), at least one monomer is used which is chosen from N-vinylimidazoles and diallylamines, optionally in partially or completely quaternized form.

Preferred examples of monomers (a) are 3-methyl-1-vinylimidazolium chloride and methosulfate, dimethyldiallylammonium chloride, and N,N-dimethylaminoethyl methacrylate and N-[3-(dimethylamino)propyl]methacrylamide which have been quaternized by methyl chloride, dimethyl sulfate or diethyl sulfate.

Particularly preferred monomers (a) are 3-methyl-1-vinylimidazolium chloride and methosulfate and dimethyldiallylammonium chloride, and very particular preference is given to 3-methyl-1-vinylimidazolium chloride and methosulfate.

Suitable water-soluble monomers (b) different from (a) are N-vinyllactams, e.g. N-vinylpiperidone, N-vinylpyrrolidone and N-vinylcaprolactam, N-vinylacetamide, N-methyl-N-vinylacetamide, acrylamide, methacrylamide, N,N-dimethylacrylamide, N-methylolmethacrylamide, N-vinyloxazolidone, N-vinyltriazole, hydroxyalkyl (meth)acrylate, e.g. hydroxyethyl (meth)acrylate and hydroxypropyl (meth)acrylate, or alkylethylene glycol (meth)acrylates having 1 to 50 ethylene glycol units in the molecule.

Also suitable are N-vinylimidazoles of the formula (I) in which R¹ to R³ are hydrogen, C₁-C₄-alkyl or phenyl, diallylamines of the formula (II), and dialkylaminoalkyl (meth)acrylates and dialkylaminoalkyl (meth)acrylamides of the formula (III), e.g. dimethylaminoethyl methacrylate or dimethylaminopropylmethacrylamide.

Also suitable are unsaturated carboxylic acids, e.g. acrylic acid, methacrylic acid, crotonic acid, itaconic acid, maleic acid, fumaric acid or their corresponding anhydrides, and unsaturated sulfonic acids, such as e.g. acrylamidomethylpropanesulfonic acid and vinylsulfonic acid.

As monomer (b), preference is given to using at least one N-vinyllactam. Very particular preference is given to N-vinylpyrrolidone.

Suitable as monomers (c) are C₁-C₄₀-alkyl esters, in particular C₁-C₂₄-, in particular C₁-C₁₀-alkyl esters of (meth)acrylic acid, the esters being derived from linear, branched-chain or carbocyclic alcohols, e.g. methyl (meth)acrylate, ethyl (meth)acrylate, tert-butyl (meth)acrylate, isobutyl (meth)acrylate, n-butyl (meth)acrylate, steryl (meth)acrylate, or esters of alkoxylated fatty alcohols, e.g. C₁-C₄₀-fatty alcohols, reacted with ethylene oxide, propylene oxide or butylene oxide, in particular C₁₀-C₁₈-fatty alcohols reacted with 3 to 150 ethylene oxide units. Also suitable are acrylamides, such as N-tert-butylacrylamide, N-butylacrylamide, N-octylacrylamide, N-tert-octylacrylamide and N-alkyl-substituted acrylamides having linear, branched-chain or carbocyclic alkyl radicals, it being possible for the alkyl radical to have the meanings given above for R⁴.

Also suitable are styrene, vinyl and allyl esters of C₁-C₄₀-carboxylic acids, which may be linear, branched-chain or carbocyclic, e.g. vinyl acetate, vinylpropionate, vinyl neononanoate, vinylneoundecanoic acid, vinyl t-butylbenzoate, alkyl vinyl ethers, for example methyl vinyl ether, ethyl vinyl ether, butyl vinyl ether, steryl vinyl ether.

As monomers (d), use is made of bi- or polyfunctional free-radically copolymerizable monomers.

Monomers (d) which have a crosslinking function are, in particular, compounds with at least 2 ethylenically unsaturated, nonconjugated double bonds in the molecule.

Suitable crosslinkers are, for example, acrylic esters, methacrylic esters, allyl ethers or vinyl ethers of at least dihydric alcohols. The OH groups of the parent alcohols may be in completely or partially etherified or esterified form; however, the crosslinkers contain at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, neopentyl glycol mono(hydroxypivalate), 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans having molecular weights of in each case 200 to 10,000. Apart from the homopolymers of ethylene oxide and/or propylene oxide, it is also possible to use block copolymers of ethylene oxide or propylene oxide, or copolymers which contain ethylene oxide and propylene oxide groups in incorporated form. Examples of parent alcohols having more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide, as the corresponding ethoxylates or propoxylates respectively. The polyhydric alcohols can also firstly be converted to the corresponding glycidyl ethers by reaction with epichlorohydrin.

Further suitable crosslinkers are the vinyl esters or the esters of monohydric unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the above-described polyhydric alcohols, for example oleic acid, crotonic acid, cinnamic acid or 10-undecenoic acid.

Also suitable as monomers (d) are straight-chain or branched, linear or cyclic, aliphatic or aromatic hydrocarbons which have at least two double bonds which in the case of aliphatic hydrocarbons must not be conjugated, e.g. divinylbenzene, divinyltoluene, 1,7-octadiene, 1,9-decadiene, 4-vinyl-1-cyclohexene, trivinylcyclohexane or polybutadienes having molecular weights of from 200 to 20,000.

Also suitable as crosslinkers are the acylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Also suitable are the amides of allylamine and unsaturated carboxylic acids such as acrylic acid, methacrylic acid, itaconic acid, maleic acid, or at least dibasic carboxylic acids.

Triallylamine and triallylmonoalkylammonium salts, e.g. triallylmethylammonium chloride or methyl sulfate, are also suitable as crosslinkers.

Also suitable are N-vinyl compounds of urea derivatives, at least difunctional amides, cyanurates or urethanes, for example of urea, ethyleneurea, propyleneurea or tartramide, e.g. N,N′-divinylethyleneurea or N,N′-divinylpropyleneurea.

Further suitable crosslinkers are divinyldioxane, tetraallylsilane or tetravinylsilane.

Preference is given to using crosslinkers which are soluble in the monomer mixture.

Particularly preferred crosslinkers are, for example, methylenebisacrylamide, triallylamine and triallylalkylammonium salts, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred as crosslinkers are pentaerythritol triallyl ether, methylenebisacrylamide, N,N′-divinylethyleneurea, tiallylamine and acrylic esters of glycol, butanediol, trimethylolpropane or glycerol, or acrylic esters of glycol, butanediol, trimethylolpropane or glycerol reacted with ethylene oxide and/or epichlorohydrin.

The monomers (a) to (d) can be used in each case individually or in a mixture with other monomers of the same group.

The preparation of the polymers can be carried out by processes of free-radically initiated polymerization known per se, e.g. by solution polymerization, emulsion polymerization, suspension polymerization, precipitation polymerization, inverse suspension polymerization or inverse emulsion polymerization or by polymerization in supercritical media, e.g. supercritical carbon dioxide, without the methods which can be used being limited thereto.

The polymerization is usually carried out at temperatures of from 20° C. to 150° C. and at atmospheric pressure or under autogenous pressure. The temperature can be kept constant or be increased continuously or discontinuously, e.g. in order to increase the conversion.

As initiators for the free-radical polymerization it is possible to use the water-soluble and water-insoluble peroxo and/or azo compounds customary for this purpose, for example alkali metal or ammonium peroxidisulfates [sic], dibenzoyl peroxide, tert-butyl perpivalate, tert-butyl per-2-ethylhexanoate, di-tert-butyl peroxide, tert-butyl hydroperoxide, azobisisobutyronitrile, azobis-(2-amidinopropane) dihydrochloride or 2,2′-azobis-(2-methylbutyronitrile). Also suitable are initiator mixtures or redox initiator systems, such as e.g. ascorbic acid/iron(II) sulfate/sodium peroxodisulfate, tert-butyl hydroperoxide/sodium disulfite, tert-butyl hydroperoxide/sodium hydroxymethanesulfinate. The initiators can be used in the customary amounts, for example 0.05 to 5% by weight, based on the amount of monomers to be polymerized.

The molecular weight and the K value of the polymers can be varied widely in a manner known per se through the choice of polymerization conditions, for example polymerization time, polymerization temperature or initiator concentration, and by the content of crosslinker. The K values of the polymers are in a range between 30 and 350, preferably 50 and 350.

The K values are measured in accordance with Fikentscher, Cellulosechemie, vol. 13, pp. 58-64 (1932) at 25° C. on 0.1% strength solutions in 0.5 molar sodium chloride solution.

The molecular weights of the polymers are generally between 5000 and 10,000,000, in particular between 10,000 and 5,000,000, preferably between 20,000 and 3,000,000.

Relaxer

The term relaxer (straightening agent, hair-smoothing agent) encompasses agents which are used for the purposes of smoothing naturally curly or artificially curled hair, and also agents which are used in the course of a permanent waving treatment.

A large number of compounds are available to the person skilled in the art as relaxers (component (ii)). In principle, all compounds which effect partial dissolution of the tertiary structure of the keratin in the hair, in particular a reduction of disulfide bridges of the cystine bonds in the hair, are suitable here.

Relaxers which may be mentioned are products based on hydroxides, such as alkali metal hydroxides (e.g. sodium hydroxide, potassium hydroxide, lithium hydroxide (so-called lye relaxers) and guanidine hydroxide (so-called no-lye relaxers). These compounds effect hydrolysis of the peptide bonds in the hair, forming lanthionine. Products based on guanidine hydroxide usually consist of two components: a cream component which, in addition to water, mineral oil, emulsifiers and fatty alcohols, comprises an alkali metal hydroxide, and an activator which comprises a concentrated solution of a guanidine compound. The alkali metal hydroxide of the cream component used is usually calcium hydroxide. The guanidine compound usually used is guanidine sulfate, sulfite, carbonate, phosphate, nitrate, acetate, bisulfate, bisulfite, hydrochloride, fluoride, oxalate, tartrate, laurate, alginate, alkane- and alkenecarboxylic acids having 2 to 20 carbon atoms. Particular preference is given to the use of guanidine carbonate. Shortly before use, these two components are mixed with one another, and guanidine hydroxide is formed. As relaxers, mention may also be made of thioglycolic acid. Also suitable are substances which contain sulfur and have a reducing action, such as, for example, products based on cysteine, cysteamine, sulfite, monoglyceryl thioglycolate, thiolactic acid or thioglycerol.

In a preferred embodiment of the present invention the relaxers used are compounds chosen from the group formed from alkali metal hydroxides, guanidine hydroxide and thioglycolic acid. Suitable as alkali metal hydroxides are, in particular, sodium hydroxide, potassium hydroxide and/or lithium hydroxide.

The content of polymer (i) in the agents according to the invention is usually between 0.01 and 10% by weight, in particular between 0.05 and 7% by weight, preferably between 0.1 and 5% by weight, based on the final preparation. Particular preference is given to a content between 0.1 and 0.5% by weight. The polymers can be used in the form of aqueous solutions.

The content of relaxer (ii) in the agents according to the invention is generally between 0.5 and 15% by weight, in particular between 1.0 and 10% by weight, preferably between 1.0 and 5.0% by weight, particularly preferably between 1.5 and 2.5% by weight, based on the final preparation, and can be chosen in a targeted manner by the person skilled in the art depending on the hair structure and the desired degree of relaxation.

The contact time of the agents according to the invention can be chosen by the person skilled in the art depending on the hair structure and the desired degree of relaxation. Customary contact times are in the range from 10 to 20 min. Following the action of the agents, they are usually rinsed out with lukewarm water. Following treatment of the hair with the agents according to the invention, the hair is in the alkaline state. It is therefore usually aftertreated with neutralizing preparations, usually slightly acidic solutions.

Compared with the products of the prior art, no relatively long contact times are required for the agents according to the invention. Furthermore, to achieve effective relaxation, it is not necessary to increase the content of relaxer. Thus, without increasing the content of relaxers, effective hair-smoothing is achieved while simultaneously avoiding scalp irritations, and with retention of the hair structure. An essential feature of the invention is the avoidance of damage to the hair structure compared with products of the prior art which generally effect aftertreatment of the hair already damaged by the relaxation. Compared with products of the prior art, the agents according to the invention can be washed out particularly readily and easily, the hair is readily combable in the wet state and, surprisingly, proves to be protected against thermal stresses, as occur, in particular, during a subsequent permanent waving treatment.

The invention therefore further provides for the use of the agents as claimed in claim 1 and/or 2 for the relaxation of hair, in particular for the smoothing of hair.

The agents according to the invention are suitable in particular for the relaxation of hair in the course of a permanent waving deformation.

The agents according to the invention can be present in end preparations as aqueous or aqueous-alcoholic solutions, O/W and W/O emulsions in the form of shampoos, creams, mousses, sprays, gels or gel sprays, and can, accordingly, be formulated with further customary auxiliaries.

Further customary auxiliaries which may be mentioned are: surfactants, oil substances, emulsifiers, coemulsifiers, superfatting agents, pearlescent waxes, bodying agents, thickeners, fats, waxes, silicone compounds, hydrotropic agents, preservatives, perfume oils, dyes, stabilizers, pH regulators, care substances such as panthenol, collagen, vitamins and proteinaceous substances, solubilizers, complexing agents and the like. Furthermore, it is possible for traditional hair cosmetic polymers different to (i) to be present.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable compounds are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate may be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is approximately 6 to 60 mols per mole of alcohol. Also suitable are alkylamine oxides, mono- or dialkylalkanolamides, fatty acid esters of polyethylene glycols, ethoxylated fatty acid amides, alkyl polyglycosides or sorbitan ether esters.

In addition, the agents can comprise customary cationic surfactants, such as e.g. quaternary ammonium compounds, for example cetyltrimethylammonium chloride.

The agents according to the invention, in particular in the form of shampoo formulations, usually comprise anionic surfactants as base surfactants, and amphoteric and nonionic surfactants as cosurfactants.

The agents usually comprise 2 to 50% by weight of surfactant, preferably 5 to 40% by weight, particularly preferably 8 to 30% by weight.

Suitable oil substances are, for example, Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of hydroxycarboxylic acids with linear or branched C₆-C₂₂-fatty alcohols, in particular dioctyl malate, esters of linear and/or branched fatty acids with polyhydric alcohols (such as e.g. propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, vegetable oils, branched primary alcohols, substituted cyclohexanes, linear and branched C₆-C₂₂-fatty alcohol carbonates, Guerbet carbonates, esters of benzoic acid with linear and/or branched C₆-C₂₂-alcohols (e.g. Finsolv® TN), linear or branched, symmetrical or unsymmetrical dialkyl ethers having 6 to 22 carbon atoms per alkyl group, ring-opening products of epoxidized fatty acid esters with polyols, silicone oils and/or aliphatic or naphthenic hydrocarbons.

Suitable emulsifiers are, for example, nonionogenic surfactants from at least one of the following groups:

(1) addition products of 2 to 30 mol of ethylene oxide and/or 0 to 5 mol of propylene oxide with linear fatty alcohols having 8 to 22 carbon atoms, with fatty acids having 12 to 22 carbon atoms and with alkylphenols having 8 to 15 carbon atoms in the alkyl group;

(2) C_{12/18}-fatty acid mono- and diesters of addition products of from 1 to 30 mol of ethylene oxide with glycerol;

(3) glycerol mono- and diesters and sorbitan mono- and diesters of saturated and unsaturated fatty acids having 6 to 22 carbon atoms and the ethylene oxide addition products thereof;

(4) alkyl mono- and oligoglycosides having 8 to 22 carbon atoms in the alkyl radical and the ethoxylated analogs thereof;

(5) addition products of from 15 to 60 mol of ethylene oxide with castor oil and/or hydrogenated castor oil;

(6) polyol and, in particular, polyglycerol esters, such as e.g. polyglycerol polyricinoleate, polyglycerol poly-12-hydroxystearate or polyglycerol dimerate. Also suitable are mixtures of compounds of two or more of these classes of substance;

(7) addition products of from 2 to 15 mol of ethylene oxide with castor oil and/or hydrogenated castor oil;

(8) partial esters based on linear, branched, unsaturated or saturated C_{6/22}-fatty acids, ricinoleic acid, and 12-hydroxystearic acid and glycerol, polyglycerol, pentaerythritol, dipentaerythritol, sugar alcohols (e.g. sorbitol), alkylglucosides (e.g. methylglucoside, butylglucoside, laurylglucoside), and polyglucosides (e.g. cellulose);

(9) mono-, di- and trialkyl phosphate, and mono-, di- and/or tri-PEG alkyl phosphates and salts thereof;

(10) wool wax alcohols;

(11) polysiloxane-polyalkyl-polyether copolymers or corresponding derivatives;

(12) mixed esters of pentaerythritol, fatty acids, citric acids and fatty alcohol according to German patent 1165574 and/or mixed esters of fatty acids having 6 to 22 carbon atoms, methylglycose and polyols, preferably glycerol or polyglycerol, and

(13) polyalkylene glycols.

The addition products of ethylene oxide and/or propylene oxide with fatty alcohols, fatty acids, alkylphenols, glycerol mono- and diesters, and sorbitan mono- and diesters of fatty acids or with castor oil are known, commercially available products. They are homolog mixtures whose average degree of alkoxylation corresponds to the ratio of the amounts of ethylene oxide and/or propylene oxide and substrate with which the addition reaction is carried out. C_{12/18}-fatty acid mono- and diesters of addition products of ethylene oxide with glycerol are known from German patent 2024051 as refatting agents for cosmetic preparations. C_{8/18}-alkyl mono- and oligoglycosides, their preparation and their use are known from the prior art. Their preparation takes place, in particular, by reacting glucose or oligosaccharides with primary alcohols having 8 to 18 carbon atoms. With regard to the glycoside ester, monoglycosides in which a cyclic sugar radical is bonded to the fatty alcohol glycosidically, and also oligomeric glycosides having a degree of oligomerization up to preferably about 8 are suitable. The degree of oligomerization here is a statistical average value which is based on a homolog distribution customary for such technical-grade products.

It is also possible for the emulsifiers used to be zwitterionic surfactants. Zwitterionic surfactants is the term used to refer to those surface-active compounds which carry at least one quaternary ammonium group and at least one carboxylate and one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines [sic] having in each case 8 to 18 carbon atoms in the alkyl or acyl group, and cocacylaminoethyl hydroxyethylcarboxymethylglycinate. Particular preference is given to the fatty acid amide derivative known under the CTFA name Cocamidopropyl Betaine. Likewise suitable emulsifiers are ampholytic surfactants. Ampholytic surfactants are understood as meaning those surface-active compounds which, apart from a C_{8/18}-alkyl or acyl group in the molecule, contain at least one free amino group and at least one —COOH— or —SO₃H— group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocacylaminoethylaminopropionate and C_{12/18}-acylsarcosine. In addition to the ampholytic emulsifiers, quaternary emulsifiers are also suitable, those of the esterquat type, preferably methyl-quaternized difatty acid triethanolamine ester salts, being particularly preferred.

As superfatting agents, it is possible to use substances such as, for example, lanolin and lecithin, and polyethoxylated or acylated lanolin and lecithin derivatives, polyol fatty acid esters, monoglycerides and fatty acid alkanolamides, the latter also serving as foam stabilizers.

Examples of suitable pearlescent waxes are: alkylene glycol esters, specific [sic] ethylene glycol disterate; fatty acid alkanolamides, specifically coconut fatty acid diethanolamide; partial glycerides, specifically stearic acid monoglyceride; esters of polybasic, optionally hydroxy-substituted carboxylic acids with fatty alcohols haviong 6 to 22 carbon atoms, specifically long-chain esters of tartaric acid; fatty substances, such as, for example, fatty alcohols, fatty ketones, fatty aldehydes, fatty ethers and fatty carbonates which have a total of at least 24 carbon atoms, specifically laurone and disteryl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Suitable bodying agents are primarily fatty alcohols or hydroxy fatty alcohols having 12 to 22 and, preferably, 16 to 18 carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, polysaccharides, in particular xanthan gum, guar guar, agar agar, alginates and Tyloses, carboxymethcellulose [sic] and hydroxyethylcellulose, and also relatively high molecular weight polyethylene glycol mono- and diesters of fatty acids, polyacrylates (e.g. Carbopols® from Goodrich or Synthalens® from Sigma), polyacrylamides, polyvinyl alcohol and polyvinylpyrrolidone, surfactants, such as, for example, ethoxylated fatty acid glycerides, esters of fatty acids with polyols, such as, for example, pentaerythritol or trimethylolpropane, fatty alcohol ethoxylates having a narrowed homolog distribution or alkyl oligoglucosides, and electrolytes, such as sodium chloride and ammonium chloride.

Typical examples of fats are glycerides, and suitable waxes are, inter alia, beeswax, carnauba wax, candelilla wax, montan wax, paraffin wax or microcrystalline waxes, optionally in combination with hydrophilic waxes, e.g. cetylstearyl alcohol or partial glycerides. Stabilizers which may be used are metal salts of fatty acids, such as e.g. magnesium, aluminum and/or zinc stearate or ricinoleate.

Suitable silicone compounds are, for example, dimethylpolysiloxanes, methylphenylpolysiloxanes, cyclic silicones, and amino-, fatty-acid-, alcohol-, polyether-, epoxy-, fluorine-, glycoside- and/or alkyl-modified silicone compounds, which can either be in liquid or resin form at room temperature. Typical examples of fats are glycerides, and suitable waxes are, inter alia, beeswax, carnauba wax, candelilla wax, montan wax, paraffin wax or microcrystalline waxes, optionally in combination with hydrophilic waxes, e.g. cetylstearyl alcohol or partial glycerides. Stabilizers which may be used are metal salts of fatty acids, such as e.g. magnesium, aluminum and/or zinc stearate. [sic]

To improve the flow behavior, it is also possible to use hydrotropic agents, such as, for example, ethanol, isopropyl alcohol or polyols. Polyols which are suitable here preferably have 2 to 15 carbon atoms and at least two hydroxyl groups. Typical examples are

gycerol;

alkylene glycols, such as, for example, ethylene glycol, diethylene glycol, propylene glycol, butylene glycol, hexylene glycol, and polyethylene glycols having an average molecular weight of from 100 to 1000 daltons; technical-grade oligoglycerol mixtures having a degree of self-condensation of from 1.5 to 10, such as, for example, technical-grade diglycerol mixtures with a diglycerol content of from 40 to 50% by weight;

methylol compounds, such as, in particular, trimethylolethane, trimethylolpropane, trimethylolbutane, pentaerythritol and dipentaerythritol;

lower alkylglucosides, in particular those having 1 to 8 carbon atoms in the alkyl radical, such as, for example, methyl- and butylglucoside;

sugar alcohols having 5 to 12 carbon atoms, such as, for example, sorbitol or mannitol;

sugars having 5 to 12 carbon atoms, such as, for example, glucose or sucrose;

amino sugars, such as, for example, glucamine.

Examples of suitable preservatives are phenoxyethanol, formaldehyde solution, parabens, pentanediol or sorbic acid, and the other classes of substance listed in Appendix 6, Part A and B, of the Cosmetics Directive.

Perfume oils which may be mentioned are mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are e.g. benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate [sic], allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, and the ketones include, for example, the ionones, cc-isomethylionone [sic] and methyl cedryl ketone, and the alcohols include anethole, citronellol, eugenol, isoeugenol, geraniol, linalool, phenylethyl alcohol and terioneol [sic], and the hydrocarbons include mainly the terpenes and balsams. Preference is, however, given to using mixtures of different fragrances which together produce a pleasing scent note. Essential oils of lower volatility, which are mostly used as flavor components, are also suitable as perfume oils, e.g. sage oil, camomile oil, oil of cloves, balm oil, mint oil, cinnamon leaf oil, lime blossom oil, juniperberry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, Romillat, Irotyl and Floramat alone or in mixtures.

Dyes which may be used are the substances approved and suitable for cosmetic purposes, as are listed, for example, in the publication “Kosmetische Färbemittel” [Cosmetic Colorants] from the Farbstoffkommission der Deutschen Forschungsgemeinschaft [Dyes Commission of the German Research Council], Verlag Chemie, Weinheim, 1984, pp. 81-106. These dyes are usually used in concentrations of from 0.001 to 0.1% by weight, based on the total mixture.

Examples of conventional hair cosmetic polymers different from (i) are anionic polymers. Such anionic polymers are homo- and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid, vinyl propionate (e.g. Luviset® CAP), maleic anhydride copolymers, optionally reacted with alcohols, anionic polysiloxanes, e.g. carboxy-functional copolymers of vinylpyrrolidone, t-butyl acrylate, methacrylic acid (e.g. Luviskol® VBM).

Very particularly preferred anionic polymers are acrylates with an acid number greater than or equal to 120 and copolymers of tert-butyl acrylate, ethyl acrylate, methacrylic acid.

Further suitable hair cosmetic polymers are e.g. copolymers of N-vinylpyrrolidone/dimethylaminoethyl methacrylate, quaternized with diethyl sulfate (Luviquat® PQ11), cationic cellulose derivatives (polyquaternium-4 and -10), acrylamide copolymers (polyquaternium-7) and guar hydroxypropyltrimethyl ammoniumchloride (INCI: Hydroxypropyl Guar Hydroxypropyltrimonium Chloride).

Also suitable as further hair cosmetic polymers are neutral polymers, such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethyleneimines and salts thereof, polyvinylamines and salts thereof, cellulose derivatives, polyaspartic acid salts and derivatives.

The total proportion of auxiliaries and additives can be 1 to 50% by weight, preferably 5 to 40% by weight, based on the composition.

### EXAMPLES

Preparation Examples Polymers

### Example 1

A mixture of 48 g of 3-methyl-1-vinylimidazolium methylsulfate, 192 g of N-vinylpyrrolidone and 350 g of water was adjusted to a pH of 7.8 using 10% strength by weight sodium hydroxide solution (feed 1). 3.0 g of 2,2′-azobis-(2-methylpropionamidine) dihydrochloride and 100 g of water were used to prepare feed 2 a [sic] 2 l stirred container which was equipped with stirrer, heating, reflux condenser and metering device was charged with 300 g of water, 100 ml of feed 1 and 12 ml of feed 2, and the mixture was heated to 60° C. with stirring. At this temperature, the remainder of feed 1 was metered in over the course of 4 hours, and the remainder of feed 2 over the course of 6 hours. The mixture was then stirred at this temperature for a further hour. This gave a clear, viscous polymer solution. The K value of the polymer was 300.4 (0.1% strength by weight).

### Example 2

A mixture of 120 g of 3-methyl-1-vinylimidazolium methylsulfate, 120 g of N-vinylpyrrolidone, 1.2 g of mercaptoethanol and 350 g of water was adjusted to a pH of 7.5 using concentrated ammonia solution (feed 1). 3.0 g of 2,2′-azobis-(2-methylpropionamidine) dihydrochloride and 100 g of water were used to prepare feed 2. A 2 l stirred container which was equipped with stirrer, heating, reflux condenser and metering device was charged with 300 g of water, 100 ml of feed 1 and 12 ml of feed 2, and the mixture was heated to 55° C. with stirring. At this temperature, the remainder of feed 1 was metered in over the course of 7 hours and the remainder of feed 2 over the course of 9 hours. The mixture was then stirred at this temperature for a further one hour. This gave a clear, viscous polymer solution. The K value of the polymer was 82.3 (1% strength by weight).

### Example 3

A mixture of 203 g of monomer solution according to Example 1, 100 g of water and 280 g of vinyl pytrolidone, referred to below as feed 1, is adjusted to a pH of 7.5 using concentrated ammonia solution. 2 g of 2,2′-azobis(2-amidinopropane) hydrochloride and 55 g of water are used to prepare a second solution, referred to below as feed 2.

362 g of water, 50 ml of feed 1 and 5 ml of feed 2 are heated to 75° C. with stirring in a 2 l glass vessel equipped with stirrer, heating, reflux condenser and metering devices. After the intended temperature has been reached, the remainder of feed 1 is metered in over the course of four hours, and the remainder of feed 2 over the course of five hours at a constant temperature of 75° C. The mixture is then stirred for a further hour at this temperature. This gives a clear, high-viscosity polymer solution. The K value of the polymer is 141.5.

### Example 4

A mixture of 483 g of monomer solution according to Example 3, 15 g of vinylpyrrolidone and 137 g of water is adjusted to a pH of 7.5 using concentrated ammonia solution (feed 1). 3 g of 2,2′-azobis-(2-amidinopropane) hydrochloride and 75 g of water are used to prepare feed 2.

A 2-1 glass vessel equipped with a stirrer, heating, reflux condenser and metering devices is charged with 290 g of water, 100 ml of feed 1 and 8 ml of feed 2, and the mixture is heated to 65° C. with stirring. At this temperature, the remainder of feed 1 is metered in over the course of 5 hours, and the remainder of feed 2 over the course of 7 hours. The mixture is then stirred for a further hour at this temperature. This gives a clear, viscous polymer solution. The K value of the polymer is 98.2.

### Example 5

Feed 1 consists of a mixture of 203 g of a monomer solution according to Example 3, 280 g of vinylpyrrolidone, 100 g of water and 1.2 g of 2-mercaptoethanol. Feed 2 is a solution of 8 g of hydrogen peroxide (30% strength, in 75 g of water). A 2 l glass vessel equipped with stirrer, heating, reflux condenser and metering devices is charged with 320 g of water, 50 ml of feed 1 and 10 ml of feed 2, and the mixture is heated to 65° C. At this temperature, the remainder of feed 1 is metered in over the course of 5 hours, and the remainder of feed 2 over the course of 7 hours. The mixture is stirred for a further hour at this temperature. This gives a clear, viscous polymer solution. The K value of the polymer is 72.5.

### Examples 6 to 8

300 g of water were charged to a stirred apparatus with attached reflux condenser and heated to 65° C. with stirring in a stream of nitrogen. After this temperature had been reached, the monomer mixture (composition see table [lacuna] in 250 g of water was added over the course of 4 hours, and a feed of 2 g of 2,2′-azobis(2-amidinopropane) dihydrochloride in 50 g of water was added over the course of 5 hours. The mixture was then stirred for a further 2 hours at this temperature. This gave a clear solution of a polymer.

TABLE

| | | N-vinyl- | N-Vinyl- | K |
|---|---|---|---|---|
| Ex. | Monomer | caprolactam | pyrrolidone | value |
| 6 | 3-Methyl-1-vinylimidazolium | 200 g | 160 g | 245 |
| | chloride 40 g | | | |
| 7 | 3-Methyl-1-vinylimidazolium | 200 g | 160 g | 275 |
| | methylsulfate 40 g | | | |
| 8 | 3-Methyl-1-vinylimidazolium | 120 g | 160 g | 256 |
| | methylsulfate 120 g | | | |

### Example 9

A stirred apparatus was charged with 400 g of water and 46 g of dimethyldiallylammonium chloride solution (65% strength). 10% of feed 1, consisting of 270 g of N-vinylpyrrolidone and 0.6 g of N,N′-divinylethyleneurea, was added to this initial charge. The mixture was heated to 60° C. with stirring in a stream of nitrogen, and feed 1 was metered in over the course of 3 hours, and feed 2, consisting of 0.9 g of 2,2′-azobis(2-amidinopropane) dihydrochloride in 100 g of water, was metered in over the course of 4 hours. After 3 hours, the mixture was diluted with 700 g of water and stirred for a further hour. 1.5 g of 2,2′-azobis(2-amidinopropane) dihydrochloride in 30 g of water were then added and the mixture was stirred for a further 2 hours at 60° C. This gave a colorless high-viscosity polymer solution with a solids content of 20.9% and a K value of 80.3.

### Example 10

A stirred apparatus was charged with 300 g of feed 1, consisting of 200 g of N-vinylpyrrolidone, 77 g of dimethyldiallylammonium chloride solution (65% strength), 1.13 g of N,N′-divinylethyleneurea and 440 g of water, and the mixture was heated to 60° C. with stirring in a stream of nitrogen. The remainder of feed 1 was metered in over the course of 2 hours, and feed 2, consisting of 0.75 g of 2,2′-azobis(2-amidinopropane) dihydrochloride in 100 g of water, was metered in over the course of 4 hours. After the end of feed 1, the reaction mixture was diluted with 1620 g of water. After the end of feed 2, the mixture was stirred for a further hour at 60° C., then 1.25 g of 2,2′-azobis(2-amidinopropane) dihydrochloride in 65 g of water were added and the mixture was stirred for a further hour. This gave a colorless high-viscosity polymer solution with a solids content of 10.2% and a K value of 80.

### Example 11

130 g of water and 48 g of 3-methyl-1-vinylimidazolium chloride were charged to a stirred apparatus and heated to 60° C. with stirring in a stream of nitrogen. Then, feed 1, consisting of 192 g of N-vinylpyrrolidone, 0.48 g of N,N′-divinylethyleneurea and 450 g of water, was metered in over the course of 3 hours, and feed 2, consisting of 1.44 g of 2,2′-azobis(2-amidinopropane) dihydrochloride in 80 g of water, was metered in over the course of 4 hours. The mixture was then stirred at 60° C. for a further hour. In order to keep the mixture stirrable, it was diluted with a total of 2100 g of water as required. This gave a colorless high-viscosity polymer solution with a solids content of 8.2% and a K value of 105.

### Example 12

716 g of water were introduced into a stirred apparatus and heated to 60° C. with stirring in a stream of nitrogen. Then, feed 1, consisting of 180 g of N-vinylpyrrolidone, 20 g of 3-methyl-1-vinylimidazolium methylsulfate, 0.32 g of N,N′-divinylethyleneurea and 25 g of water, was metered in over the course of 2 hours, and feed 2, consisting of 0.6 g of 2,2′-azobis(2-amidinopropane) dihydrochloride in 60 g of water, was metered in over the course of 3 hours. After the end of feed 1, the reaction mixture was diluted with 1000 g of water. After feed 2, the mixture was stirred at 70° C. for a further 3 hours. This gave a colorless high-viscosity polymer solution with a solids content of 11.0% and a K value of 86.

### Example 13

440 g of water were introduced into a stirred apparatus and heated to 60° C. with stirring in a stream of nitrogen. Then, feed 1, consisting of 180 g of N-vinylpyrrolidone, 20 g of 3-methyl-1-vinylimidazolium methylsulfate, 0.30 g of N,N′-divinylethyleneurea and 25 g of water, was metered in over the course of 2 hours, and feed 2, consisting of 0.6 g of 2,2′-azobis(2-amidinopropane) dihydrochloride in 60 g of water, was metered in over the course of 3 hours. After feed 2, the mixture was stirred at 70° C. for a further 3 hours. In order to keep the reaction mixture stirrable, it was diluted with a total of 1275 g of water as required. This gave a colorless high-viscosity polymer solution with a solids content of 11.3% and a K value of 105.

### Example 14

650 g of water were introduced into a stirred apparatus and heated to 60° C. with stirring in a stream of nitrogen. Then, feed 1, consisting of 225 g of N-vinylpyrrolidone, 25 g of 2,3-dimethyl-1-vinylimidazolium methylsulfate, 0.25 g of N,N′-divinylethyleneurea and 580 g of water, was metered in over the course of 3 hours, and feed 2, consisting of 0.7 g of 2,2′-azobis(2-amidinopropane) dihydrochloride in 100 g of water, was metered in over the course of 4 hours. After the end of feed 1, the reaction mixture was diluted with 835 g of water. After feed 2, the mixture was stirred for a further hour and 1.25 g of 2,2′-azobis(2-amidinopropane) dihydrochloride in 77 g of water were then metered in. The mixture was then stirred at 70° C. for a further 2 hours. This gave a colorless high-viscosity polymer solution with a solids content of 10.4% and a K value of 106.

### Example 15

650 g of water were introduced into a stirred apparatus and heated to 60° C. with stirring under a stream of nitrogen. Then, feed 1, consisting of 225 g of N-vinylpyrrolidone, 25 g of 2,3-dimethyl-1-vinylimidazolium methylsulfate, 0.375 g of N,N′-divinylethyleneurea and 580 g of water, was metered in over the course of 3 hours, and feed 2, consisting of 0.7 g of 2,2′-azobis(2-amidinopropane) dihydrochloride in 100 g of water, was metered in over the course of 4 hours. After the end of feed 1, the reaction mixture was diluted with 1135 g of water. After feed 2, the mixture was stirred for a further hour and 1.25 g of 2,2′-azobis(2-amidinopropane) dihydrochloride in 77 g of water were then metered in. The mixture was then stirred at 70° C. for a further 2 hours. This gave a colorless high-viscosity polymer solution with a solids content of 9.2% and a K value of 92.

### Example 16

A reaction vessel with nitrogen blanketing was charged with 800 g of cyclohexane, 5 g of sorbitan monooleate, 5 g of Hypermer B246¹ and 1 g of 2,2′-azobis(2,4-dimethylvaleronitrile), and the mixture was heated to 65° C. The feed, consisting of 100 g of 3-methyl-1-vinylimidazolium methylsulfate, 100 g of N-vinylpyrrolidone, 100 g of water and 0.25 g of tripropylene glycol diacrylate, was metered in over the course of 20 minutes. The mixture was then stirred at 65° C. for six hours. Then, 200 g of cyclohexane were added, and the water was distilled off azeotropically, and the polymer was filtered off and dried. The K value of an aqueous solution of the polymer was 114. ¹ Hypermer B246: polymeric surfactant from ICI

### Example 17

900 g of ethyl acetate were introduced into a stirred apparatus and heated to 77° C. with stirring in a stream of nitrogen. Then, feed 1, consisting of 270 g of N-vinylpyrrolidone, 30 g of 1-vinylimidazole and 0.3 g of N,N′-divinylethyleneurea, was metered in over the course of 3 hours, and feed 2, consisting of 3 g of 2,2′-azobis(2-methylbutyronitrile) in 80 g of ethyl acetate, was metered in over the course of 4 hours. The mixture was then stirred for a further 2 hours and cooled to room temperature, and 36 g of dimethyl sulfate were added. The mixture was then stirred for half an hour at room temperature and for a further 2 hours at 70° C. The resulting powder was filtered off and dried. The K value of an aqueous solution of the polymer was 125.

### Example 18

440 g of water were introduced into a stirred apparatus and heated to 60° C. with stirring in a stream of nitrogen. Then, feed 1, consisting of 144 g of N-vinylpyrrolidone, 16 g of 3-methyl-1-vinylimidazolium methylsulfate, 1.4 g of tetraethylene glycol diacrylate and 100 g of water, was metered in over the course of 2 hours, and feed 2, consisting of 0.8 g of 2,2′-azobis(2-amidinopropane) dihydrochloride in 50 g of water, was metered in over the course of 3 hours. After feed 2, the mixture was stirred for a further 3 hours at 70° C. In order to keep the reaction mixture stirrable, it was diluted with a total of 1200 g of water as required. This gave a colorless high-viscosity polymer solution with a solids content of 8.5% and a K value of 95.

### Example 19

550 g of water were introduced into a stirred apparatus and heated to 60° C. with stirring in a stream of nitrogen. Then, feed 1, consisting of 102 g of N-vinylpyrrolidone, 26 g of 3-methyl-1-vinylimidazolium methylsulfate, 0.8 g of triallylamine and 100 g of water, was metered in over the course of 2 hours. Feed 2, consisting of 0.6 g of 2,2′-azobis(2-amidinopropane) dihydrochloride in 50 g of water, was added to the reaction mixture over the course of 3 hours. After feed 2, the mixture was stirred for a further 3 hours at 70° C. In order to keep the reaction mixture stirrable, it was diluted with a total of 1000 g of water as required. This gave a slightly yellowish, high-viscosity polymer solution with a solids content of 7.0% and a K value of 102.

### Example 20

Example 11 was repeated, except that 2.2 g of pentaerythritol triallyl ether were used instead of triallylamine. This gave a slightly yellowish, high-viscosity polymer solution with a K value of 95.

### Example 21

440 g of water were introduced into a stirred apparatus and heated to 60° C. with stirring in a stream of nitrogen. Then, feed 1, consisting of 150 g of N-vinylpyrrolidone, 8 g of 3-methyl-1-vinylimidazolium methylsulfate, 0.6 g of triallylamine and 100 g of water, was metered in over the course of 2 hours, and feed 2, consisting of 0.8 g of 2,2′-azobis(2-amidinopropane) dihydrochloride in 50 g of water, was metered in over the course of 3 hours. After feed 2, the mixture was stirred for a further 3 hours at 70° C. In order to keep the reaction mixture stirrable, it was diluted with a total of 1200 g of water as required. This gave a colorless high-viscosity polymer solution with a solids content of 8.1% and a K value of 98.

### Example 22

A reaction vessel with nitrogen blanketing was charged with 800 g of cyclohexane, 5 g of sorbitan monooleate and 5 g of Hypermer B246², and the mixture was heated to 60° C. Feed 1, consisting of 60 g of 3-methyl-1-vinylimidazolium methylsulfate, 140 g of N-vinylpyrrolidone, 150 g of water and 1.0 g of triallylamine, and feed 2, consisting of 0.6 g of 2,2′-azobis(2-amidinopropane) dihydrochloride in 50 g of water, were metered in over the course of 1 hour. The mixture was then stirred at 60° C. for a further 6 hours. 200 g of cyclohexane were then added and the water was distilled off azeotropically, and the polymer was filtered off and dried. ²Hypermer B24® [sic] polymeric surfactant from ICI

### Example 23

A reaction vessel with nitrogen blanketing was charged with 800 g of cyclohexane, 5 g of sorbitan monooleate and 5 g of Hypermer B246³ and the mixture was heated to 60° C. Feed 1, consisting of 20 g of 3-methyl-1-vinylimidazolium methylsulfate, 180 g of N-vinylpyrrolidone, 150 g of water and 0.5 g of triallylamine, was metered in over the course of 1 hour, and feed 2, consisting of 1.2 g of 2,2′-azobis(2-amidinopropane) dihydrochloride in 70 g of water, was metered in over the course of 4 hours. The mixture was then stirred for a further 3 hours at 60° C. 200 g of cyclohexane were then added and the water was distilled off azeotropically, and the polymer was filtered off and dried.

### Example 24

A stirred apparatus was charged with 400 g of water, 100 g of N-vinylpyrrolidone, 11 g of 3-methyl-1-vinylimidazolium methylsulfate and 0.4 g of triallylamine, and the mixture was heated to 60° C. with stirring in a stream of nitrogen. Then, feed 1, consisting of 0.6 g of 2,2′-azobis(2-amidinopropane) dihydrochloride in 50 g of water, was added to the reaction mixture over the course of 3 hours and diluted with 1000 g of water. The mixture was then stirred for a further 3 hours at 80° C. This gave a colorless high-viscosity polymer solution with a solids content of 7.6% and a K value of 110.

### FORMULATION EXAMPLES

The viscosity of the examples was determined in accordance with Brookfield at 25° C., RVD II, Spindel 2.

### Formulation Example 1

Hair-Smoothing Preparation Comprising Sodium Hydroxide

| | | % by | |
|---|---|---|---|
| Phase | INCI name (Trade name) | wt. | Function |
| A | Ceteareth-6 (Cremophor ® A 6) | 2.0 | Emulsifier |
| | Ceteareth-25 (Cremophor ® A 25) | 2.0 | Emulsifier |
| | Cetearyl alcohol | 8.0 | Emulsifier |
| | Polyoxyethylene lanolin | 1.0 | Conditioner |
| B | Demineralized water | 51.23 | |
| | Disodium EDTA | 0.2 | Complexing agent |
| | Propylene glycol | 3.0 | Solvent |
| | Polymer according to Ex. 9 | 7.0 | Polymer (i) |
| | (6.5% by weight aqueous solution) | | |
| | (Luviquat Care ®) | | |
| | Citric acid | 0.5 | pH adjustment |
| C | Mineral oil and | 5.0 | Thickener |
| | Styrene isoprene/butadiene | | |
| | copolymer | | |
| | Cetearyl octanoate (Luvitol EHO) | 0.5 | Refatting agent |
| D | Demin. water | 17.07 | |
| | Sodium hydroxide | 2.5 | Relaxer (ii) |

³ Hypermer B24 ® [sic] polymeric surfactant from ICI

Preparation:

Phases A and B are heated separately from one another to about 80° C. Phase B is then added to Phase A with homogenization. With further homogenization, phase C is added. The emulsion is cooled to about 40° C., then phase D is added, and the mixture is homogenized again. The viscosity of the resulting composition is 12,000 mPas.

### Formulation Example 2

| | | % by | |
|---|---|---|---|
| Phase | INCI name (Trade name) | wt. | Function |
| A | Ceteareth-6 | 2.0 | Emulsifier |
| | (Cremophor ® A 6) | | |
| | Ceteareth-25 (Cremophor ® A 25) | 2.0 | Emulsifier |
| | Cetearyl alcohol | 8.0 | Emulsifier |
| | Mineral oil | 5.0 | |
| B | Demineralized water | 77.3 | |
| | Disodium EDTA | 0.2 | Complexing agent |
| | Propylene glycol | 3.0 | Solvent |
| | Polymer as in Examples 1 or 2 | 2.5 | Polymer (i) |
| | Polyquaternium-44 | | |
| | (6.5% by weight aqueous solution) | | |
| | Sodium hydroxide | 2.5 | Relaxer (ii) |

Preparation:

Phases A and B are heated separately from one another to about 80° C. Phase B is then added to Phase A with homogenization. After cooling to room temperature, the pH is adjusted to pH=12 with sodium hydroxide solution. The viscosity of the resulting composition is 9000 mPas.

### Formulation Example 3

Pump Spray

| | % by |
|---|---|
| INCI name (Trade name) | wt. |
| Cocotrimonium methosulfate | 10.0 |
| (Luviquat mono LS; 30% strength aqueous solution of | |
| lauryl/myristylmethylammonium methosulfate) | |
| Polymer as in Ex. 1 or 2 | 2.5 |
| Polyquaternium-44 (Luviquat Care ®) | |
| 6.5% strength by weight aqueous solution | |
| Disodium EDTA | 0.2 |
| Sodium hydroxide (pH = 12) | q.s. |
| Water | ad 100 |

Preparation:

The components are mixed with stirring until a clear solution forms, and adjusted to pH=12 with sodium hydroxide.

### Formulation Example 4

Two-Component System Containing Guanidine Hydroxide

Component 1

| | | % by | |
|---|---|---|---|
| Phase | INCI name (Trade name) | weight | Function |
| A | Ceteareth-6 (Cremophor ® A 6) | 2.0 | Emulsifier |
| | Ceteareth-25 (Cremophor ® A 25) | 2.0 | Emulsifier |
| | Cetearyl alcohol | 8.0 | Emulsifier |
| | Mineral oil | 5.0 | |
| B | Demineralized water | 72.3 | |
| | Disodium EDTA | 0.2 | Complexing agent |
| | Propylene glycol | 3.0 | Solvent |
| | Polymer as in Examples 9 to 24 | 2.5 | |
| | Polyquaternium-44 | | |
| | (Luviquat Care ®) | | |
| | 6.5% strength by weight of | | |
| | aqueous solution | | |
| | Calcium hydroxide | 5.0 | Component 1 of |
| | | | the relaxer |

Preparation of Component 1:

Phases A and B are heated separately from one another to about 80° C. Phase B is then added to Phase A with homogenization. The pH is 12.2.

Component 2

| | | % by | |
|---|---|---|---|
| Phase | INCI name (Trade name) | wt. | Function |
| A | Demineralized water | 74.8 | |
| | Xanthan gum | 0.2 | Thickener |
| | Cetearyl alcohol | 8.0 | Emulsifier |
| B | Guanidine carbonate | 25.0 | Component 2 of |
| | | | the relaxer |

Phase A is heated to about 80° C. and then cooled to 40° C. Phase B is then added with stirring. The pH is 11.6.

For use, component 1 is mixed with component 2 [lacuna] ratio 2:1.

### Formulation Example 5

Hair-Smoothing Composition Comprising Thioglycolic Acid

| | | % by |
|---|---|---|
| Phase | INCI name (Trade name) | wt. |
| A | Ceteareth-6 (Cremophor ® A 6) | 2.0 |
| | Ceteareth-25 (Cremophor ® A 25) | 2.0 |
| | Cetearyl alcohol | 8.0 |
| | Mineral oil | 5.0 |
| B | Disodium EDTA | 0.2 |
| | Propylene glycol | 3.0 |
| | Polymer as in Examples 9 to 24 | 2.5 |
| | Polyquaternium-44 (Luviquat Care ®) | |
| | 6.5% strength by weight aqueous solution | |
| | Thioglycolic acid 99% | 9.0 |
| | Ammonia (aqueous 25% strength by weight solution) | 12.0 |
| | Demineralized water | ad 100 |

Preparation:

Phases A and B are heated separately from one another to about 80° C; Phase B is then added to Phase A with stirring and homogenized.

### Formulation Example 6

Hair-Smoothing Composition Comprising Thioglycolic Acid

| | % by |
|---|---|
| INCI name (Trade name) | wt. |
| Cocotrimonium methosulfate | 10.0 |
| (Luviquat Mono LS; 30% strength aqueous solution of | |
| lauryl/myristylmethylammonium methosulfate) | |
| Disodium EDTA | 0.2 |
| Polymer as in Examples 9 to 24 | 2.5 |
| Polyquaternium-44 (Luviquat Care ®) | |
| 6.5% strength by weight aqueous solution | |
| Thioglycolic acid 99% | 12.0 |
| Ammonia (aqueous 25% strength by weight solution) | 25.0 |

### Formulation Example 7

Fixative for Formulation Example 6

| | | % by |
|---|---|---|
| Phase | INCI name (Trade name) | weight |
| A | Cocotrimonium methosulfate | 10.0 |
| | (Luviquat Mono LS; 30% strength aqueous solution | |
| | of lauryl/myristylmethylammonium methosulfate) | |
| | Perfume | 0.2 |
| | PEG-40 Hydrogenated castor oil (Cremophor ® RH 40) | 2.0 |
| B | Potassium bromate | 5.0 |
| | Polymer as in Examples 9 to 24 | 2.5 |
| | Polyquaternium-44 (Luviquat Care ®) | |
| | 6.5% strength by weight of aqueous solution | |
| | Disodium EDTA | 0.2 |
| | Preservative | 0.5 |
| | Citric acid (pH 4-5) | q.s. |
| | (aqueous solution of citric acid) | |
| | Demineralized water | 80.3 |

Preparation:

Phases A and B are mixed separately from one another, then Phase B is added to Phase A. The pH is adjusted to 3-3.5 with an aqueous citric acid solution.

### Formulation Example 8

Fixative for Formulation Example 6

| | | % by |
|---|---|---|
| Phase | INCI name (Trade name) | weight |
| A | Ceteareth-6 (Cremophor ®A 6) | 2.0 |
| | Ceteareth-25 (Cremophor ®A 25) | 2.0 |
| | Cetearyl alcohol | 8.0 |
| | Mineral oil | 5.0 |
| B | Propylene glycol (1,2 Propylene glycol USP) | 3.0 |
| | Potassium bromate | 5.0 |
| | Polymer as in Examples 9 to 24 | |
| | (Polyquaternium-44) (Luviquat Care ®) | 2.5 |
| | 6.5% strength by weight of aqueous solution | |
| | Disodium EDTA | 0.2 |
| | Preservative | 0.5 |
| | Citric acid (pH 3-3.5) | q.s. |
| | (aqueous solution of citric acid) | |
| | Demineralized water | ad 100 |
| C | Perfume | 0.2 |

Preparation:

Phases A and B are heated separately from one another to about 80° C. Phase B is then added to Phase A with homogenization. After cooling to about 40° C., Phase C is added. The pH is adjusted to pH=3-3.5 with citric acid.

The fixatives (Formulation examples 7 and 8) are used with the hair-smoothing composition (Formulation example 6) in the ratio 1:1.

Measurements on the Hair

1. Measurement of the Zeta Potential

Samples: Afro-American hair

a) untreated

b) treated for 15 min with a 2.2% by weight aqueous sodium hydroxide solution

c) [lacuna] for 15 min with a 2.2% by weight aqueous sodium hydroxide solution +0.5% by weight polymer as in Example 9 (Luviquat Care®)

Following treatment, the hair was neutralized to pH=6 with an aqueous solution of citric acid. The zeta potential was then measured. For this, the streaming potential of hair is measured in an electrokinetic analyzer (EKA) (Anton Paar GmbH). Measurement parameters 1 mM KCl solution, pH 7, 20° C.

The measurement produced the following results: a) −32 mV, b) −50 mV, c) +8 mV

As the results show, treatment of the hair with NaOH leads to a lowering of the zeta potential (corresponds to increased damage to the hair). Treatment of the hair with the compositions according to the invention results in a significant increase in the zeta potential, which mirrors a considerable improvement in the structure of the hair.

2. Half-Head Test

To investigate the relaxation and also the scalp irritation and hair structure, a half-head test was carried out on five subjects. For this, in each case one half of the scalp was treated with Formulation example 1 and one half of the scalp was treated with Formulation example 1 without polymer without Luviquat Care® for 15 min. The preparation was then rinsed out using a neutralization solution (citric acid solution pH 3). The parameters evaluated were the hair-smoothing action, irritation to the scalp during treatment, the rinse-off behavior, the structure and manageability of the hair after treatment (combability, ability to be blow dried etc.) both by the hairdressers and by the subjects. The table below gives the evaluation by the subjects of the side of the hair treated with the preparation according to the invention compared with the control side. The number in brackets is the number of subjects with this rating. The rating was graded as follows:

“++” = significantly better, “+” = better, 0 = no difference,
“−” worse; “− − ” significantly worse

| | Hair-Treatment |
|---|---|
| | Preparation as in |
| Parameter | Formulation example 1 |
| Scalp irritation | “++” (2); “0” (3) |
| Combability after neutralization | “++” (4); “0” (1) |
| Structure of the hair 3 days | “++” (5) |
| after the treatment | |

3. Swelling of the Hair

Laser scanning microscopy was used to analyze the swelling of the hair. For this, individual strands of hair were treated over a period of from 5 to 40 min with a 2.2% strength by weight NaOH solution (comparison), and also a 2.2% strength by weight NaOH solution containing 0.5% by weight of polymer as in Example 9 (composition according to the invention). The swelling was given in % based on the untreated hair. As can be seen from the table below, using the compositions according to the invention it is possible to achieve a significant reduction in the swelling of the hair (and thus damage of the structure).

TABLE
Swelling of Afro-American hair
Indication of swelling in [%] compared with untreated hair

| | Time (min) | | | | | |
|---|---|---|---|---|---|---|
| | 0 | 5 | 10 | 20 | 30 | 40 |
| Blank value (water) | 0 | 0 | 0 | 2.63 | 2.63 | 2.63 |
| Comparison | 0 | 11.42 | 28.57 | 34.29 | 40.0 | 42.86 |
| (2.2% strength by weight | | | | | | |
| NaOH solution) | | | | | | |
| Example | 0 | 3.03 | 18.18 | 21.21 | 27.27 | 36.36 |
| 2.2% strength by weight | | | | | | |
| NaOH solution containing | | | | | | |
| 0.5% by weight of | | | | | | |
| polymer as in Example 9 | | | | | | |

We claim:

1. A hair cosmetic agent comprising
(i) polymer obtainable by free-radically initiated copolymerization of
(a) 1 to 99.99% by weight of at least one cationic monomer optionally in partially or completely quaternized form,
(b) 5 to 97.99% by weight of at least one water-soluble monomer different from (a),
(c) 0 to 50% by weight of at least one further free-radically copolymerizable monomer different from (a) or (b) and
(d) 0.01 to 10% by weight of at least one di- or polyfunctional free-radically copolymerizable monomer different from (a), (b) or (c) and
subsequent partial or complete quaternization or protonation of the polymer if a nonquaternized or only partially quaternized monomer is used as monomer (a),
(ii) relaxers.

2. An agent as claimed in claim 1, wherein, as monomer (a), at least one monomer is used chosen from N-vinylimidazoles and diallylamines, optionally in partially or completely quaternized form.

3. An agent as claimed in claim 1 and/or 2, wherein, as monomer (a), at least one N-vinylimidazole derivative of the formula (I) is used 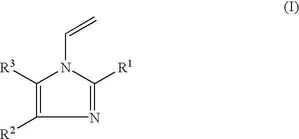
in which R¹ to R³ independently of one another are hydrogen, C₁-C₄-alkyl or phenyl.

4. An agent as claimed in claim 1 and/or 2, wherein, as monomer (a), at least one diallylamine derivative of the formula (II) is used, 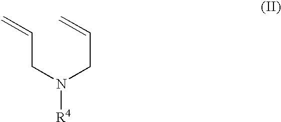
in which R⁴ is C₁-C₂₄-alkyl.

5. An agent as claimed in claim 1, wherein, as monomer (b), at least one N-vinyllactam is used.

6. An agent as claimed in at least one of the preceding claims, wherein the relaxer (ii) is chosen from the group formed from alkali metal hydroxides, guanidine hydroxides and thioglycolic acid.

7. An agent as claimed in claim 6, wherein the relaxer used is sodium hydroxide, potassium hydroxide and/or lithium hydroxide.

8. An agent as claimed in claim 1, which comprises
0.01 to 10% by weight of (i)
0.5 to 15% by weight of (ii)
—based on the final preparation—.

9. The use of agents as claimed in claim 1 for the relaxing of hair, in particular for hair smoothing.

10. The use as claimed in claim 9 for the relaxing of hair in the course of a permanent wave deformation.
